(19) United States
(12) Patent Application Publication
(10) Pub. No.: US 2024/0339218 A1
(43) Pub. Date: Oct. 10, 2024

(54) SYSTEMS AND METHODS FOR SEPSIS DETECTION AND MANAGEMENT IN PATIENTS

(71) Applicant: Deepull Diagnostics S.L., Barcelona (ES)

(72) Inventors: Jordi CARRERA FABRA, Barcelona (ES); Rafael BRU GIBERT, Barcelona (ES); Richard Max IVEY, Parkton, MD (US)

(73) Assignee: Deepull Diagnostics S.L., Barcelona (ES)

(21) Appl. No.: 18/681,425

(22) PCT Filed: Aug. 3, 2022
(86) PCT No.: PCT/US2022/039290

§ 371 Date: Feb. 5, 2024

Related U.S. Application Data
(60) Provisional application No. 63/230,436, filed on Aug. 6, 2021.

(51) Int. Cl. G16H 50/20; C12Q 1/18; C12Q 1/686; C12Q 1/689; G01N 21/65; G16B 25/20; G16B 40/10; G16H 10/60; G16H 20/10

(52) U.S. Cl. CPC G16H 50/20; C12Q 1/18; C12Q 1/686; C12Q 1/689; G01N 21/65; G16B 25/20; G16B 40/10; G16H 10/60; G16H 20/10

(57) ABSTRACT

A sepsis detection system includes a first subsystem configured to detect a presence of an infection in a patient, a second subsystem configured to detect a presence of a dysregulated host response in the patient, a third subsystem configured to detect organ dysfunction in the patient, a fourth subsystem configured to detect antimicrobial resistance (AMR) of a pathogen in the patient, and a processing device. The first, second, third, and fourth subsystems and the processing device are communicatively coupled together via a network. The processing device is configured to determine a presence of sepsis in the patient based on the presence of the infection, the presence of the dysregulated host response, and clinical data indicative of the organ dysfunction in the patient. The subsystems utilize at least one of polymerase chain reaction (PCR) processing. Raman spectroscopy, clinical data, electronic health record (EHR) data, and antimicrobial susceptibility testing (AST).

### BACKGROUND

### Field

The present disclosure relates to systems, methods, and devices for early sepsis recognition, detection, and management by utilizing one or more of Raman spectroscopy, polymerase chain reaction (PCR), single cell microscopy, and artificial intelligence (AI) techniques.

### Background

Sepsis is defined as life-threatening organ dysfunction caused by a dysregulated host response to infection. A patient experiencing or undergoing sepsis may start with a local infection, such as pneumonia, that results in inflammation of the body from the patient's immune system going into overdrive. Inflammation may ultimately lead to organ failure and death of the patient if left untreated. Sepsis causes 11 million deaths annually, and many of these cases can be prevented by early diagnosis and proper clinical management. Detecting sepsis early, when it can still be treated efficiently, is essential to improve patient outcomes, reduce morbidity and mortality.

In some cases, it may be difficult to identify sepsis because of the lack of a single specific biomarker for sepsis detection. There may be several different biomarkers or indicators of sepsis present in a patient at various times, such as procalcitonin (PCT), C-reactive protein (CRP), Further, such biomarkers for early recognition and diagnosis of sepsis might not be sensitive and specific enough to detect sepsis before symptoms appear. Their positive predictive value may be a limiting factor in many cases. Thus, it may be challenging to point to one particular criteria that is needed for sepsis detection due to the complexities and rapid progression of the sepsis response in patients.

Sepsis diagnosis may be performed by running laboratory tests to culture blood samples for identifying infection. Current testing times may take several days to obtain results from a laboratory as a result of the time needed for obtaining and processing blood cultures for sepsis-causing bacteria to ultimately determine its susceptibility to effective antimicrobials for treatment. However, sepsis detection may be time-sensitive for patients in a hospital, because sepsis can lead to septic shock and death within hours if not properly identified and treated in time. Additionally, blood culture testing is slow and might not consistently provide reliable results of bacteria or fungi detection in patients who are clinically suspected of having sepsis, especially for patients already undergoing antibiotic therapy. There is often a low yield for obtaining positive blood cultures for patients, and patients may be experiencing sepsis even without the identification of a positive blood culture.

### BRIEF SUMMARY

Embodiments of the present invention provide a cost-effective solution for improved diagnostic methods, systems, and devices for detecting sepsis much earlier in the disease cascade, identifying pathogens and antimicrobial susceptibility, and managing appropriate treatments in patients for better patient outcomes.

Described herein are systems, methods, and devices for early sepsis recognition, detection, and management by utilizing training learning algorithms, Raman spectroscopy, clinical data, electronic health record (EHR) data, and polymerase chain reaction (PCR) technology. The present disclosure provides systems, methods, and devices for rapid immune (host) response detection (or characterization), pathogen identification, and antimicrobial susceptibility testing (AST) directly from blood samples, or other samples, such as urine, sterile body fluids, or the like. Some embodiments provide an early-warning functionality for monitoring EHR data of patients, along with a Raman spectroscopy device that scans blood samples to identify patients with a high likelihood of having an infection, or developing sepsis or septic shock.

Some embodiments also identify pathogens without a culture step using a multiplex PCR approach, significantly reducing the analysis time. Some embodiments may also utilize PCR techniques to detect genotypic resistance information of a pathogen. Single cell microscopy may be leveraged to identify phenotypical susceptibility of the pathogen, leading to a diagnostic pathway for rapid and effective antimicrobial treatments.

Systems and methods include the prediction of sepsis conditions and its severity based on the interrogation of several elements of information available-including clinical data, immune response data, and infection data. This data may also be used for the stratification of patients based on different information available.

In an embodiment, an example system is described. The system includes a first subsystem configured to detect a presence of an infection in a patient, a second subsystem configured to detect a presence of a dysregulated host response in the patient, a third subsystem configured to detect organ dysfunction in the patient, and a processing device. The first subsystem, the second subsystem, the third subsystem, and the processing device are communicatively coupled together via a network. The processing device is configured to determine a presence of sepsis in the patient based on the presence of the infection, the presence of the dysregulated host response, and clinical data indicative of the organ dysfunction in the patient.

In another embodiment, an example system is described. The system includes a first subsystem configured to detect a presence of an infection in a patient, a second subsystem configured to detect a presence of a dysregulated host response in the patient, a third subsystem configured to detect organ dysfunction in the patient, a fourth subsystem configured to detect antimicrobial resistance (AMR), such as susceptibility of a pathogen in the patient from a sample, and a processing device. The first subsystem, the second subsystem, the third subsystem, the fourth subsystem, and the processing device are communicatively coupled together via a network. The processing device is configured to determine a presence of sepsis in the patient based on the presence of the infection, the presence of the dysregulated host response, and clinical data indicative of the organ dysfunction in the patient.

In yet another embodiment, and example system is described. The system includes a first subsystem configured to receive a first sample of the patient, a second subsystem configured to obtain Raman spectrum data of the patient from the first sample, and a processing device. The processing device is configured to acquire one or more patient variables from electronic health record (EHR) data for the patient, receive the Raman spectrum data of the patient from the second subsystem, and classify the patient in an immune profile group by applying a trained learning algorithm to at least one of the EHR data and the Raman spectrum data.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate embodiments of the present disclosure and, together with the description, further serve to explain the principles of the disclosure and to enable a person skilled in the pertinent art to make and use the disclosure.

FIG. 1A illustrates an example diagram of a sepsis detection workflow, according to embodiments of the present disclosure.

FIG. 1B illustrates an example diagram of a sepsis detection system, according to embodiments of the present disclosure.

FIG. 2 illustrates an example diagram of a Raman spectroscopy device in the sepsis detection system, according to embodiments of the present disclosure.

FIG. 3 illustrates an example diagram of a processing device in the sepsis detection system, according to embodiments of the present disclosure.

FIG. 4 illustrates an example diagram of an analyzer in the sepsis detection system, according to embodiments of the present disclosure.

FIG. 5 illustrates an example flowchart diagram of a method for training a learning algorithm for identifying sepsis in patients, according to embodiments of the present disclosure.

FIG. 6 illustrates an example flowchart diagram of a method for determining a likelihood of sepsis in a patient, according to embodiments of the present disclosure.

FIG. 7 illustrates a block diagram of example components of a computer system, according to embodiments of the present disclosure.

Embodiments of the present disclosure will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that this disclosure can also be employed in a variety of other applications.

It is noted that references in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

### Introduction

Sepsis is defined as life-threatening organ dysfunction caused by a dysregulated immune (host) response to infection. Sepsis may typically be caused by a bacterial infection, but can also be caused by fungi and virus. The sepsis cascade may start with infection, followed by an uncontrolled or dysregulated immune response to the infection, which ultimately results in organ dysfunction, organ failure, and/or death. In some embodiments, a patient undergoing sepsis may exhibit symptoms of systemic inflammatory response syndrome (SIRS), which may progress to sepsis, septic shock, multiple organ dysfunction (MOD), and ultimately death. As sepsis worsens, it may cause abnormal blood coagulation, which results in small clots or burst blood vessels that damage or destroy tissues. This affects blood flow to vital organs such as the brain, heart, and kidneys, resulting in organ dysfunction and damage. In the hospital setting, there is an urgent need to identify patients in danger of sepsis and manage care of these patients appropriately. Without accurate diagnosis and appropriate therapy, the likelihood of patient morbidity and mortality increases dramatically by the hour for every hour.

Rapid sepsis detection methods today are limited. The standard of care for detecting sepsis relies on blood culture, for which the average time to detection is about 13 hours. Blood culture testing provides an organism, without identification (ID) of the pathogen, followed by plating of positives on petri dishes. In a conventional blood culture process, two blood culture sets are taken per adult patient, in which each set consists of an aerobic bottle and an anaerobic bottle to assure that the entire spectrum of sepsis causative bacteria is captured during the culture event. Generally, each culture is acquired from a separate venipuncture (e.g., left arm and right arm of the patient). This is to assure that the bacterial shedding event is captured by the culture so that the bacteria may be “recovered” for downstream testing (e.g., ID and AST). Following the culturing, the aerobic and anaerobic bottles are incubated in a blood culture instrument where they are monitored in real-time for growth. The aerobic and anaerobic bottles are incubated and agitated until any bacteria is allowed to go through a lag-log growth transition that is detected electronically. A laboratory worker may then be alerted that a positive culture exists for the patient. Typically, a blood culture will become positive in an average of about 13 hours for most bacteria, while some yeasts and fungi may take much longer (e.g., up to 5 days). However, many cultures are negative due to collection error, an insufficient blood volume taken during collection, transport delays to the lab, insufficient sensitivity, or the like.

Due to the urgent nature of sepsis, following positivity, a laboratory may immediately commence a work-up to identify the bacterial gram stain (e.g., gram positive or gram negative), determine a significant organism or contaminant, single microbe or polymicrobic infection, and report the intermediate information to the caregiver. Further, the lab may take immediate steps to identify the bacteria using rapid methods such as molecular diagnostics systems, which may take 1.5 hours to provide results. These systems may offer limited molecular information on genetic drug resistance information of certain bacteria that exhibit these profiles. Alternatively, the lab may process the positive blood culture (PBC) aliquot with a matrix-assisted laser desorption/ionization time of flight (MALDI-TOF) mass spectrometry system to report an ID in about one hour. Using the ID, the caregiver may confirm or potentially adjust antibiotics that may have been administered to the patient prophylactically. However, by the time the bacteria has been identified, up to 20-24 hours (at best) may have already passed since the patient was first cultured.

In parallel to obtaining and reporting the identified pathogen, antimicrobial susceptibility testing (AST) may also be performed to determine the antibiotic susceptibility profile of the pathogen. In some cases, AST may be performed in vitro on an isolated bacterial pathogen using manual methods including microbroth dilution methods or culture-based assays (e.g., disc-diffusion antibiotic susceptibility test or using plated petri dish media). In other cases, AST may be performed using automated devices with antimicrobial resistance panels to test for minimum inhibitory concentration (MIC) of antimicrobials or drug resistance.

However, these systems may require isolated bacterial colonies. For example, current methods for AST may necessitate a significant biomass of clean bacteria to operate properly. The subculture from the positive blood culture first has to be grown out on a plated media petri dish. This process may take 6-12 hours for a round-the-clock operating lab, but may sometimes take up to 24 hours for a lab that is closed overnight. The culture may then need to be adjusted for uniformity (e.g., 10,000 cells taken for input) before being inoculated into the AST system. Following loading, the AST analysis can take between 8-16 hours to report antimicrobial susceptibility or resistance information for all organism classes. Additionally, AST might not begin until at least day 2 of a patient's stay in the hospital. In other words, results from the AST analysis (e.g., including whether the bacteria is susceptible, intermediate, or resistant (SIR) to particular antibiotics, MIC, or resistance information) may be reported to the caregiver about 2.5-3 days after an initial collection of the original blood culture sample from the patient.

The overall process may take several days to report critical antibiotic information for sepsis patients. For example, if an infection is suspected, a sample of blood, urine, sputum, or the like, is collected from the patient and provided to a clinical laboratory to first determine if an infectious agent is present. This may require 18-24 hours (e.g., day 1) for most pathogenic bacterial species to sufficiently grow. If a bacterium is isolated, it further requires an additional 18-24 hours (e.g., day 2) to culture the isolate and another 2-48 hours (e.g., day 3+) to identify the bacterial isolate and perform AST. Traditional and automated AST methods require a “pure culture” of the bacterial isolate, along with a prolonged period (e.g., 6-24 hours) of incubation for growth of the organism. Conventional systems may be limited because they are growth-based, slow, expensive, require manual manipulation, and are not integrated. Without improved solutions for sepsis detection, patients may continue to suffer and often becomes worse as physicians treat with empiric antibiotics while awaiting more actionable information from the laboratories about the one or more causative agents for the infection (e.g., pathogens).

Current technologies do not provide an integrated and comprehensive solution for the entire workflow of host response detection, pathogen identification, and antimicrobial or antibiotic susceptibility testing (AST). In some cases, some systems focus solely on identifying a single aspect of the sepsis cascade, such as detection of host response or detection of a pathogen. For example, a system may look at host response for early indication of sepsis by detecting molecular white cell RNA markers (via reverse transcription (RT)-PCR to detect gene expression), but would not provide answers for pathogen identification or susceptibility. The immune response result may alert caregivers that a patient is entering or has entered the sepsis cascade and is in urgent need of treatment or intervention to prevent further probability of irreversible morbidity. Caregivers may immediately react by looking for the infection site and the infectious agent using traditional methods, such as by obtaining blood cultures from the infection site to identify the infectious agent.

On the pathogen detection side, current technologies may offer a direct from blood detection and identification method, using PCR from blood samples and followed by nuclear magnetic resonance (NMR) spectroscopy detection. However, such systems may be expensive, limited in menu options, and difficult to service without providing a solution for rapid AST results, instead offering a limited molecular genetic resistance panel. Other systems may utilize direct-from-blood pathogen rRNA RT-PCR for pathogen identification but may also be constrained by a limited menu (e.g., 15 targets or less). Ultimately, current technologies do not provide an automated direct-from-blood rapid AST solution, and instead rely on positive blood cultures for testing which can take 13-20 hours to report anything actionable. For example, some systems may obtain aliquots from positive blood culture (PBC) bottles, thus saving the time needed to grow the bacterial isolate from the PBC bottle (e.g., 6-24 hours). These systems, however, are limited on the numbers of drugs and organisms they can report on and thus have limited utility for healthcare providers. In order to greatly reduce morbidity and mortality, new sepsis diagnostic methods, devices, and systems are needed for rapidly determining antibiotic sensitivity and antibiotic resistance to an infectious sepsis-causing bacteria at the single cell or low copy number level, which is detected directly from a blood sample without the significant time delay required for the multiple culture steps (e.g., biological amplification) currently required by the standard of care methods.

As septic conditions may often go undetected in patients and quickly progress into life-threatening conditions, there is a clear need and demand for new and comprehensive systems, devices, and methods for acquiring rapid and relevant information on host immune response, identifying causative pathogens, and providing guidance on appropriate antimicrobial agents in order to save lives and reduce antimicrobial resistance (AMR). The systems, devices, and methods described herein provide a holistic and systematic approach for identifying and managing sepsis and its severity in patients, determining antibiotic sensitivity and resistance to identified pathogens, and recommending treatments that are effective and appropriate for patients.

### Overview of Sepsis Detection:

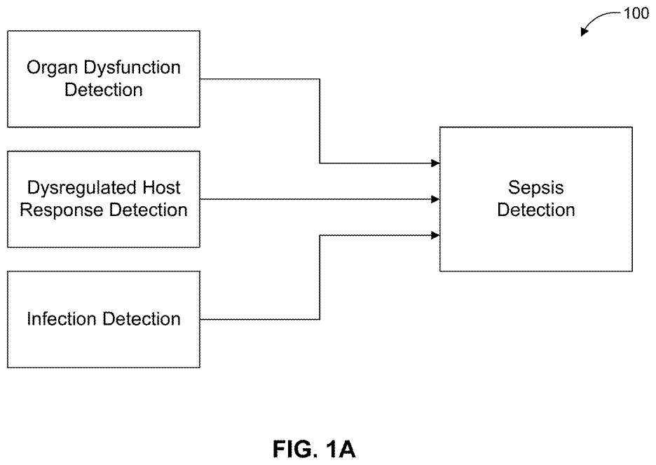

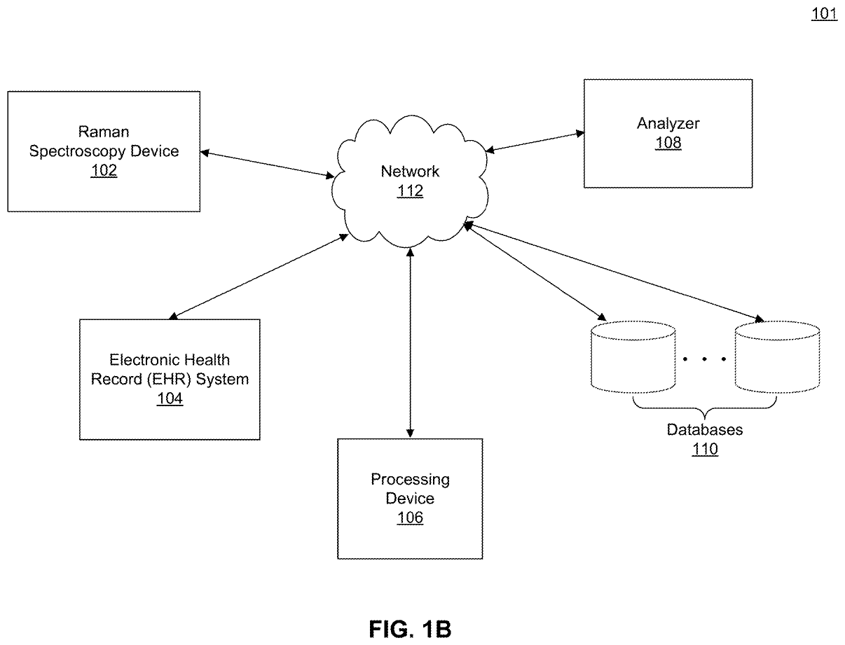

FIG. 1A illustrates an example diagram of a sepsis detection workflow 100, according to embodiments of the present disclosure. As shown by the workflow 100, sepsis may be detected by identifying three stages occurring in a patient-organ dysfunction, dysregulated host response, and infection.

Organ dysfunction may represent a condition in a patient where one or more organs are unable to perform expected functions. In some embodiments, organ dysfunction may lead to organ failure, in which organs in different systems of the body may fail as a consequence of sepsis and septic shock (e.g., multiple organ dysfunction syndrome (MODS)). In some embodiments, a caregiver of a patient might not identify sepsis occurring until the patient exhibits signs of organ dysfunction and/or organ failure. For example, signs of organ dysfunction or failure may include a fever, irregular or rapid heart rate (tachycardia), abnormally rapid breathing (tachypnea), a decrease in urine output, and the like. Because of the rapid progression of the sepsis cascade in a patient, identifying sepsis at this stage may be too late to prevent deterioration of the patient. It may be difficult for the caregiver to quickly provide the patient with proper care and treatment to prevent patient morbidity and mortality.

Organ dysfunction may ultimately result from a dysregulated host response. In some embodiments, a dysregulated host response may be referred to herein as a dysregulated immune response. The dysregulated host response may be the body's uncontrolled response to an infection or injury, in which the body does not follow a healthy immune response process. The dysregulated host response may include inflammation, immunosuppression as well as neuroendocrine, coagulation, and metabolic responses. Systemic inflammatory conditions may result from an interaction between a pathogenic microorganism and the host's defense system that triggers an excessive and dysregulated response in the host.

Identification of the dysregulated host response occurring in a patient may allow detection of the presence of infection in the patient. In particular, the dysregulated host response in a patient may be caused by an infection. In some embodiments, infection in a patient may be caused by a pathogen. In some embodiments, a pathogen may be referred to herein as a bacterium or bacteria, organism, microorganism, single-cell or single microorganism, microbe, virus, or the like. An infection may begin anywhere in the body and may spread throughout if not properly treated. For example, an infection in a patient may lead to a dysregulated host response and may ultimately cause organ dysfunction.

In some embodiments, the presence of sepsis in a patient may be determined by detecting at least one of organ dysfunction, dysregulated host response, and infection. In some embodiments, the three stages or aspects of sepsis may be detected in any order and by using various technologies as further described herein.

In some embodiments, organ dysfunction in a patient may be determined based on at least one of clinical data/EHR data, SOFA scores, and Raman spectroscopy data of a patient sample, along with trained learning algorithms. In some embodiments, a dysregulated host response in a patient may be detected by at least one of Raman spectroscopy data of a patient sample and EHR data, along with trained learning algorithms. In some embodiments, a presence of infection in a patient may be determined by at least one of performing PCR on a sample of the patient and analyzing data related to an immune response of the patient.

In some embodiments, organ dysfunction, dysregulated host response, and infection may be represented by a clinical score, immune score, and infection score, respectively. In some embodiments, the three different scores may be inputs to calculating a sepsis score for sepsis detection.

In some embodiments, organ dysfunction, dysregulated host response, and infection may detected by various subsystems and systems including different modules, computing devices or systems, and/or technologies, as further described herein.

### System Overview:

FIG. 1B illustrates an example diagram of a sepsis detection system 101, according to embodiments of the present disclosure. In some embodiments, the sepsis detection system 101 may be referred to herein as system 101. The system 101 may comprise a Raman spectroscopy device 102, electronic health record (EHR) system 104, processing device 106, analyzer 108, and a plurality of databases 110 communicatively coupled via a network 112.

In some embodiments, various combinations of one or more of the components of the system 101 may be utilized together and/or separately for detecting sepsis and identifying the progression of sepsis, including detecting organ dysfunction, dysregulated host response, and infection in a patient, as described herein.

The system 101 may comprise a Raman spectroscopy device 102 configured to scan samples of patients. In some embodiments, the Raman spectroscopy device 102 may be referred to herein as a Raman reader or Raman scanner. The Raman spectroscopy device 102 may be used to acquire Raman spectrum data for performing an immune response screening or triaging of samples of suspected sepsis patients that may enter a hospital via the emergency room (ER) or are residents within the hospital. In some embodiments, the Raman spectroscopy device 102 may scan patients' plasma samples that are preserved with ethylenediamine tetraacetic acid (EDTA). In some embodiments, the Raman spectroscopy device 102 may reside in a laboratory inside or outside of a hospital and may use a sample collection tube for reading plasma samples of patients.

In some embodiments, the Raman spectra data acquired from patients' samples by the Raman spectroscopy device 102 may be used to provide a rapid indication of a sepsis likelihood or a probability score of a patient's “sepsis state” to inform caregivers to escalate the sepsis treatment and follow a full diagnostic protocol based on the likelihood value. In particular, the Raman spectroscopy device 102 may perform Raman spectroscopy on a small portion of a plasma sample to obtain a Raman spectrum comprising one or more peaks representing a host response signature of the sample. In some embodiments, the Raman spectroscopy device 102 may communicate with the processing device 106 in the sepsis detection system 101 to use the acquired Raman spectrum data along with machine learning algorithms and/or other decision tool parameters to rapidly detect sepsis and/or provide a sepsis likelihood value or score. In some embodiments, the Raman spectroscopy device 102 may transmit Raman spectrum data to the processing device 106, and the processing device 106 may train a deep learning algorithm to identify a likelihood of infection, or sepsis in future patients using the Raman spectrum data. Ultimately, the Raman spectrum data may be used to distinguish sepsis patients from non-sepsis patients based on their Raman host response signature.

In addition to the Raman spectroscopy device 102, the system 101 further comprises an EHR system 104. The EHR system 104 may include any number of servers, computers, and/or devices that are configured to electronically store patient healthcare information. In some embodiments, the EHR system 104 may aggregate data from various healthcare services and healthcare providers, such as hospitals, clinical care facilities, laboratories, radiology providers, and pharmacies. While only one EHR system 104 is illustrated in FIG. 1B for reference, there may be any number of EHR systems 104, in which each EHR system 104 is associated with one or more hospitals or other healthcare service center.

In some embodiments, EHR system 104 may comprise one or more EHR databases (not shown) that store patient data and medical history data regarding the health and treatment of patients. In some embodiments, the data stored in the EHR system 104 may be referred to herein as EHR data. In some embodiments, one or more EHR databases in the EHR system 104 may store records for each patient, including demographics, medical history, medication and allergies, immunization status, laboratory test results, radiology images, vital signs, personal statistics like age and weight, and billing information for each patient. Records in the EHR system 104 may include records of observational data, patient encounters, lab results, prescriptions, messages (e.g., messages transmitted to patients from their providers), and also biographical information about a patient, such as name, address, date of birth, and the like. In some embodiments, the EHR system 104 may also store data regarding preexisting illnesses, long-standing comorbidities, medications, interventions, and the like for each patient.

In some embodiments, one or more of the plurality of databases 110 in system 101 may be integrated within EHR system 104. For example, the plurality of databases 110 may include one or more EHR databases, which may be accessed by processing device 106 for retrieving, acquiring, and/or monitoring patient information. In other embodiments, the plurality of databases 110 may be separate from the EHR system 104. In some embodiments, the plurality of databases 110 may represent any number of databases, and may include various databases that store clinical parameters data, epidemiology information or antibiotic resistance information for a plurality of pathogens, organ dysfunction data, Raman spectrum data, and the like. In some embodiments, the plurality of databases 110 may store organ dysfunction data for a plurality of patients, in which the organ dysfunction data includes one or more scores associated with at least one of sequential organ failure assessment (SOFA), quick SOFA, logistic organ dysfunction system (LODS), national early warning score (NEWS), and/or modified early warning score (MEWS). In some embodiments, the plurality of databases 110 may include a clinical database storing Raman spectrum data of validated sepsis specimens. For example, the clinical database may include Raman spectrum data from septic patients showing peaks that represent septic host response signatures of a plurality of samples corresponding to the septic patients. In some embodiments, the data in the clinical database or a Raman spectrum database may be collected and compiled from the Raman spectroscopy device 102 and/or the EHR system 104 by the processing device 106, and updated using one or more machine learning algorithms as described herein. In some embodiments, the clinical database may store a Raman spectral library of Raman spectrum data of known samples from septic patients.

Processing device 106 may coordinate communication, computing, and processing of data obtained from the Raman spectroscopy device 102, EHR system 104, analyzer 108, and the plurality of databases 110. In some embodiments, the processing device 106 may acquire EHR data and data associated with laboratory information system (LIS) parameters from the EHR system 104 and/or databases 110. In some embodiments, the processing device 106 may monitor EHR data and acquire patient data for one or more patient variables from the EHR data for a plurality of patients. In some embodiments, patient variables may comprise at least one of temperature, heart rate, systolic blood pressure, respiratory rate, white blood cell count, platelet count, ratio of arterial oxygen partial pressure to fractional inspired oxygen (PaO2/FiO2), bilirubin level, Glasgow Coma Scale score, cardiovascular mean arterial pressure, creatinine level, urine output, lactate level, C-reactive protein level, and procalcitonin level. The processing device 106 may monitor the EHR data and identify one or more patient variables that are indicative of a change in a condition of a patient based on the monitoring.

In some embodiments, the processing device 106 may also collect and/or receive Raman spectrum data for a plurality of patients from the Raman spectroscopy device 102. In some embodiments, the processing device 106 may use the Raman spectrum data or the EHR data separately to determine a presence of sepsis in patients by training a learning algorithm and applying the training learning algorithm to the Raman or EHR data. In additional or alternative embodiments, the processing device 106 may integrate the acquired EHR data with the acquired Raman spectrum data from the Raman spectroscopy device 102 to improve the prediction of whether or not a patient has likelihood of sepsis. In some embodiments, integration of the EHR data with Raman spectrum data may help bolster the certainty of the sepsis prediction model (e.g., the learning algorithm) of whether or not a patient being tested has a high probability or likelihood of having sepsis.

In some embodiments, the processing device 106 may further generate one or more notifications indicating results of the likelihood of sepsis prediction or identification, and the processing device 106 may transmit the one or more notifications to at least one of a computing device associated with a healthcare provider or the analyzer 108. In some embodiments, the results of the sepsis prediction or identification may indicate a low likelihood of infection and/or sepsis in the patient, in which a value of the likelihood may be less than a predetermined threshold value. In other embodiments, the results of the sepsis prediction or identification may indicate a high likelihood of infection and/or sepsis in the patient, in which a value of the likelihood may be greater than or equal to a predetermined threshold value.

In some embodiments, the processing device 106 may employ deterministic modeling to reduce false positives and unnecessary “noise” coming from systemic inflammatory response syndrome (SIRS) in patients or non-sepsis patients that may be sick with other conditions for which the learning algorithm might not be trained. Additionally, the processing device 106 may include decision models for an early warning screening of patients using Raman spectrum data (e.g., Raman scattering data) of plasma samples and Raman spectrum data from clinical databases, which may be regularly updated with validated sepsis specimens. In some embodiments, Raman spectroscopy device 102, EHR system 104, and/or processing device 106 may be utilized together to provide an early warning detection of sepsis of patients. The processing device 106 may screen patients based on Raman spectrum data and/or EHR data and help pinpoint which patients are exhibiting signs of early sepsis.

In some embodiments, the processing device 106 may monitor EHR data for a plurality of patients in the EHR system 104 and determine whether one or more patient variables are indicative of a change in a patient's condition for each patient in the plurality of patients based on the monitoring. If the values of one or more patient variables are above a predetermined threshold value for a patient, then the processing device 106 may transmit a command to the Raman spectroscopy device 102 to obtain Raman spectrum data for the identified patient. The processing device 106 may then use the collected Raman spectrum data and/or the EHR data to identify a likelihood of infection and/or sepsis in the patient, such as by applying a trained algorithm to the collected Raman spectrum data and/or the EHR data.

In some embodiments, this early warning screening implemented by the processing device 106 may identify patients are in danger of sepsis, transmit alerts to caregivers on the identified patients, and transmit notifications to computing devices in a laboratory (e.g., inside or outside of a hospital) to obtain additional specimens from the patients for diagnosis using the analyzer 108. The processing device 106 may also provide caregivers with rapid results on immune status of a patient, identification of a pathogen, and antimicrobial susceptibility testing (AST) results as information is received from one or more of the Raman spectroscopy device 102, EHR system 104, analyzer 108, and the plurality of databases 110 in the sepsis detection system 101. Additionally, as many sepsis patients are often re-admitted months after leaving the hospital, the processing device 106 may be configured to collect, process, and/or compare historical information on metrics for a sepsis patient to identify improved treatment plans for treating a recurring infection or other complications as a result of a prior sepsis event.

Upon detecting a likelihood of sepsis in a sample of a patient using the Raman spectroscopy device 102, EHR system 104, and/or processing device 106, the patient's sample (or another sample collected from the patient) may subsequently undergo direct testing by the analyzer 108 for rapid direct-from-blood identification and AST.

The analyzer 108 may include one or more modules for sample collection, pathogen identification, and susceptibility testing. In some embodiments, the analyzer 108 may employ enhanced polymerase chain reaction (PCR) technology for rapid identification of pathogens in blood samples to drive sensitivity to highly competitive levels (with respect to blood culture detection levels) while assuring specificity. In some embodiments, the analyzer 108 may use PCR technology to test a suite of viral targets or a pan-virus target in order to guide caregivers on viral vs bacterial infections or co-infections. In some embodiments, the analyzer 108 may use PCR technology to test a suite of fungal targets or a pan-fungal target. By applying PCR to samples, the analyzer 108 may provide high sensitivity for detection of low bacterial loads at the clinical level without the need for a blood culture or growth step. In some embodiments, the analyzer 108 may also incorporate PCR technology for rapid resistance detection in addition to the pathogen identification.

Additionally, the analyzer 108 may be configured to perform susceptibility testing of pathogen causing an infection in a patient. Antibiotic susceptibility testing may provide a clinician with therapeutic options to treat patients with bacterial or other microbiological infections. In some embodiments, the analyzer 108 may be configured to perform susceptibility tests for a plurality of antibiotics or antimicrobials to determine to which antibiotic or antimicrobial the pathogen is sensitive or resistant. In some embodiments, the analyzer 108 may determine minimum inhibitory concentrations (MICs) of an antibiotic that inhibit growth of a pathogen. The results of the AST may be used by the analyzer 108 and/or by the processing device 106 to determine which antimicrobials to recommend for treatment of infection in a patient.

In some embodiments, the analyzer 108 may be configured to receive a sample container or consumable that contains a sample of a patient. In some embodiments, the sample of the patient may be pipetted or transferred directly into the consumable by a user of the analyzer 108. In some embodiments, the consumable may be inserted into the analyzer 108 by the user. In some embodiments, the consumable may be disposable after a single use or reusable for testing of additional samples.

In some embodiments, the analyzer 108 may transmit results of the pathogen identification and the AST assay to the processing device 106. The processing device 106 may generate a recommendation for an antimicrobial for treatment of the patient based on the results. In some embodiments, the analyzer 108 may transmit results of the pathogen identification and the AST assay to the processing device 106, and the processing device 106 may generate a recommendation for an antimicrobial for treatment of the patient based on the results. In some embodiments, the processing device 106 may access one or more databases 110 for epidemiology information or antibiotic resistance information for the identified pathogen, and generate a recommendation for treatment of the patient based on the results of the AST assay and the epidemiology information or antibiotic resistance information for the pathogen.

In some embodiments, the Raman spectroscopy device 102, electronic health record (EHR) system 104, processing device 106, analyzer 108, and/or the plurality of databases 110 may further be coupled to one or more computing devices (not shown) associated with a healthcare provider, such as a physician, physician's assistant, nurse practitioner, nurse, clinical pharmacist, specialist, or the like. The one or more computing devices associated with the healthcare provider may be a personal digital assistant, desktop workstation, laptop or notebook computer, netbook, tablet, smart phone, mobile phone, smart watch or other wearable, or any combination thereof. In some embodiments, a healthcare provider may access results from any of the sepsis detection methods described herein using the one or more computing devices. In some embodiments, the one or more computing devices associated with the healthcare provider may transmit requests for data from the Raman spectroscopy device 102, electronic health record (EHR) system 104, processing device 106, analyzer 108, and/or the plurality of databases 110.

In some embodiments, the one or more computing devices associated with the healthcare provider may receive data regarding a sepsis status of a patient based on the detection methods performed by the Raman spectroscopy device 102, processing device 106, and/or analyzer 108. In some embodiments, the one or more computing devices associated with the healthcare provider may receive notifications regarding an identified pathogen in a patient and/or recommendations for treatment of the patient using one or more antibiotics based on susceptibility testing results from at least one of the Raman spectroscopy device 102, processing device 106, and analyzer 108.

In some embodiments, the sepsis detection system 101 may be configured to monitor EHR data for a patient using the EHR system 104 and identify a likelihood of infection or sepsis in the patient based on applying a trained learning algorithm to at least one of the EHR data and data regarding the one or more patient variables indicative of a change in the patient's condition using the processing device 106. The sepsis detection system 101 may further be configured to identify a pathogen in a sample of the patient and identify antibiotic susceptibility and/or antimicrobial resistance for the patient based on the identification of the pathogen using the analyzer 108. In some embodiments, the sepsis detection system 101 may receive organ dysfunction data for the patient from one or more databases 110, in which the organ dysfunction data includes one or more scores associated with at least one of sequential organ failure assessment (SOFA), quick SOFA, or NEWS. The sepsis detection system 101 may use the organ dysfunction data in the identification of the likelihood of infection and/or sepsis in the patient. Ultimately, the sepsis detection system 101 may provide an end-to-end integrated system for sepsis prediction or detection, pathogen identification, and susceptibility testing in order to deliver rapid results to healthcare providers for improving patient care and outcomes.

In some embodiments, the components in the sepsis detection system 101 may be communicatively coupled via network 112. In particular, the network 112 may allow transmission of information and communication between Raman spectroscopy device 102, electronic health record (EHR) system 104, processing device 106, analyzer 108, and/or the plurality of databases 110. Network 112 may be any one or any combination of a LAN (local area network), WAN (wide area network), telephone network, wireless network, point-to-point network, star network, token ring network, hub network, or other appropriate configuration. The network may comply with one or more network protocols, including an Institute of Electrical and Electronics Engineers (IEEE) protocol, a 3rd Generation Partnership Project (3GPP) protocol, a 4^{th} generation wireless protocol (4G) (e.g., the Long Term Evolution (LTE) standard, LTE Advanced, LTE Advanced Pro), a fifth generation wireless protocol (5G), and/or similar wired and/or wireless protocols, and may include one or more intermediary devices for routing data between Raman spectroscopy device 102, electronic health record (EHR) system 104, processing device 106, analyzer 108, and/or the plurality of databases 110.

### Raman Spectroscopy Device:

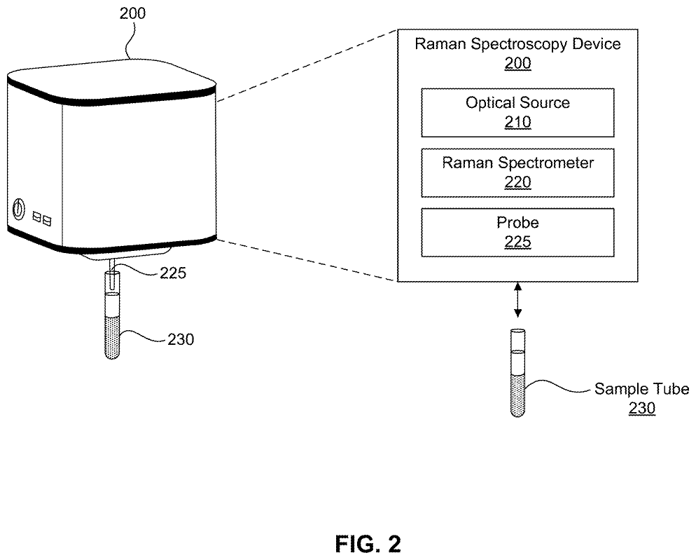

FIG. 2 illustrates an example diagram of a Raman spectroscopy device 200 in the sepsis detection system, according to embodiments of the present disclosure. The Raman spectroscopy device 200 represents an exemplary embodiment of Raman spectroscopy device 102 in FIG. 1B. The Raman spectroscopy device 200 includes an optical source 210, a Raman spectrometer 220, and a probe 225. While the Raman spectroscopy device 200 in FIG. 2 only shows three components for reference, any number of additional components for performing Raman spectroscopy may be included in the Raman spectroscopy device 200. In some embodiments, Raman spectroscopy device 200 may include additional components and/or optical elements such as lenses, filters, mirrors, gratings, detectors, objectives, and/or the like. While only one optical source 210 is illustrated in FIG. 2 for reference, there may be any number of optical sources 210 in the Raman spectroscopy device 200.

In some embodiments, the optical source 210 may be designed to output a beam of radiation at only a single wavelength, or it may be a swept source and be designed to output a range of different wavelengths. In some embodiments, the optical source 210 may comprise a laser or a laser diode. The laser may be configured to emit a radiation beam of laser energy at a wavelength of about or close to 780 nm or 532 nm. In some embodiments, two optical sources 210 may be used in the Raman spectroscopy device 200, in which the two optical sources 210 are configured to emit radiation beams at 780 nm and 532 nm, respectively.

In some embodiments, the radiation beam may be generated by the optical source 210 and transmitted through a probe 225 to a sample in the sample tube 230. The sample tube 230 may contain a plasma sample of a patient that is to be interrogated by the probe 225 to detect a likelihood of sepsis in the patient. In some embodiments, the probe 225 may comprise a fiber-optic probe configured to transmit the radiation beam into the sample. In some embodiments, the probe 225 may be configured to interact with the sample in the sample tube 230 by interrogating the sample with the transmitted radiation beam and receiving Raman scattered radiation from the sample. In some embodiments, the probe 225 may collect the returning beam from the sample tube 230 after the interrogating radiation beam has interacted with the sample.

In some embodiments, the probe 225 may be configured to route the collected radiation to the Raman spectrometer 220. In some embodiments, the collected radiation may be processed or filtered by one or more optical elements during transmission of the collected radiation from the probe 225 to the Raman spectrometer 220. In some embodiments, the Raman spectrometer 220 may be configured to receive the collected radiation beam (e.g., the Raman scattered radiation) from the probe 225. In some embodiments, the Raman spectrometer 220 may include a detector (not shown) that is configured to obtain a Raman signal from the collected radiation beam. In some embodiments, the detector may include a charge-coupled device (CCD) or a photomultiplier tube (PMT) that is highly sensitive to detect the Raman signal. In some embodiments, the detector may include a diffraction grating, either transmission or reflective.

In some embodiments, the Raman spectroscopy device 200 may include a processor (not shown) that may be configured to receive the Raman signal from the Raman spectrometer 220 and perform processing of the Raman signal to generate Raman spectrum data of the sample. In some embodiments, the Raman spectrum data may include one or more peaks representing a host response signature of the sample. In some embodiments, the Raman spectrum data may provide information on baseline parameters of a patient's immunological profile, as well as information on specific parameters directly related to the sepsis cascade, including identifying procalcitonin (PCT) and C-reactive protein (CRP) as indicators of an infection, identifying a cytokine storm as an indicator of a dysregulated host response, and identifying bilirubin and creatinine as indicators of organ failure.

The processor of the Raman spectrometer 220 may obtain the Raman spectrum data of the sample and compare the reading with previous Raman spectrum data of samples from known septic patients to identify a patient's septic status (e.g., pre-septic or at varying severity levels of sepsis). In some embodiments, the processor in the Raman spectroscopy device 200 may access a Raman spectral library that may be stored in a memory in the Raman spectroscopy device 200 or in one or more databases 110 in the sepsis detection system 101. The processor in the Raman spectroscopy device 200 may use data from the Raman spectral library to determine a likelihood of sepsis in a patient by comparing the current Raman spectrum data with historical Raman data stored in the Raman spectral library.

In additional or alternative embodiments, the Raman spectroscopy device 200 may be communicatively coupled to a separate computing device comprising a processor configured to receive the Raman signal from the Raman spectrometer 220 and perform processing of the Raman signal to generate Raman spectrum data of the sample. In some embodiments, the separate computing device may be separate from the Raman spectroscopy device 200 and may be coupled to the Raman spectroscopy device 200 via a wired or wireless connection. In some embodiments, the separate computing device coupled to the Raman spectroscopy device 200 may be the same as or different from the processing device 106 in the sepsis detection system 101.

In some embodiments, the Raman spectroscopy device 200 may be configured to perform contactless Raman spectroscopy readings on liquid plasma samples of patients. In some embodiments, the plasma used for testing might not involve any sample preparation step. In some embodiments, the probe 225 may be configured to perform a Raman reading inside the primary tube (e.g., sample tube 230) in which plasma separation of the patient sample by centrifugation is conducted. In some embodiments, the plasma may be contained in a cuvette or tube (e.g., sample tube 230) with a size and dimensions that do not affect the Raman signal reading to avoid an expensive Raman-free signal substrate.

In some embodiments, the Raman spectroscopy device 200 may be used as a standalone device for sepsis detection and management of patient samples to triage patients that may be in danger of sepsis. In other embodiments, the Raman spectroscopy device 200 may be used in combination with or integrated in a sepsis diagnostic device. For example, the Raman spectroscopy device 200 may be integrated within analyzer 108 to form a diagnostic apparatus that is configured to conduct Raman spectroscopy readings of samples and subsequently perform pathogen identification and susceptibility testing through the respective components.

Around 90% of sepsis cases are present upon admission of the patients to a hospital. Thus, in some embodiments, the Raman spectroscopy device 200 may use Raman spectrum data in combination with a plurality of diagnostic parameters collected from patients (e.g., in an emergency room (ER) of a hospital), to further improve accuracy of sepsis detection in patients. In some embodiments, the Raman spectroscopy device 200 may receive diagnostic parameter data of patients tested in an ER from the EHR system 104. In some embodiments, the diagnostic parameters obtained from the EHR system may include measured hemogram data including data on red blood cells (RBC), white blood cells (WBC), platelet count, hemoglobin, and hematocrit, coagulation data including prothrombin time, activated partial thromboplastin time, and fibrinogen, and biochemistry data including data on urea, creatinine, sodium, potassium, aspartate aminotransferase (AST) or glutamic oxaloacetic transaminase (GOT), alanine aminotransferase, and total bilirubin. In some embodiments, the Raman spectroscopy device 200 may combine the Raman spectrum data with results from other diagnostic tests (e.g., measurements of biomarkers) or additional information available in a patient's health record to narrow the results and carry out patient stratification in different phenotypes.

### Processing Device:

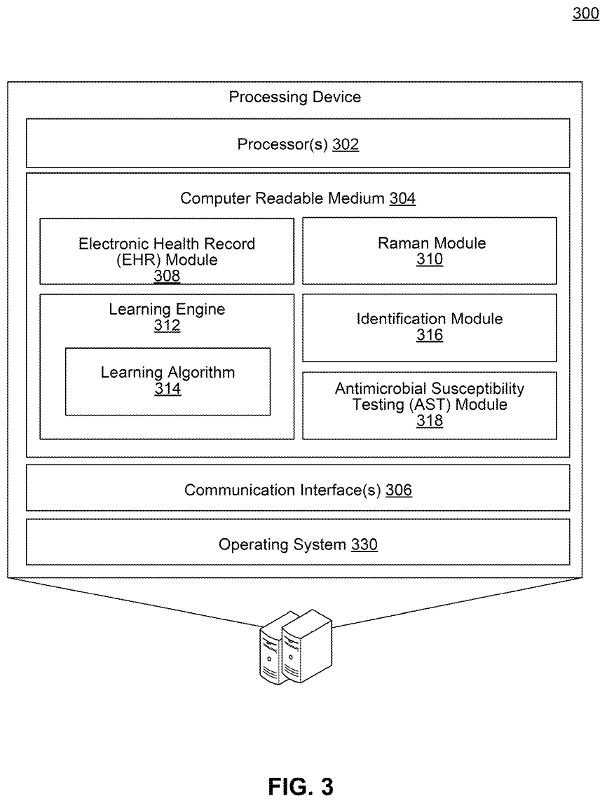

FIG. 3 illustrates an example diagram of a processing device 300 in the sepsis detection system, according to embodiments of the present disclosure. Processing device 300 represents an exemplary embodiment of processing device 106 in FIG. 1B. In some embodiments, processing device 300 may be referred to herein as a sepsis detection processing device 300.

In some embodiments, processing device 300 includes one or more computing devices that can be embodied in any number of ways. For instance, the modules, other functional components, and data can be implemented on a single computing device, a cluster of computing device, a server farm or data center, a cloud-hosted computing service, and so forth, although other computer architectures can additionally or alternatively be used.

Further, while the figures illustrate the components and data of the processing device 300 as being present in a single location, these components and data may alternatively be distributed across different computing devices and different locations in any manner. Consequently, the functions may be implemented by one or more computing devices, with the various functionality described above distributed in various ways across the different computing devices.

In the illustrated example, the processing device 300 includes one or more processors 302, one or more computer-readable media 304, and one or more communication interfaces 306. Each processor 302 is a single processing unit or a number of processing units, and may include single or multiple computing units or multiple processing cores. The processor(s) 302 can be implemented as one or more microprocessors, microcomputers, microcontrollers, digital signal processors, central processing units, state machines, logic circuitries, and/or any devices that manipulate signals based on operational instructions. For instance, the processor(s) 302 may be one or more hardware processors and/or logic circuits of any suitable type specifically programmed or configured to execute the algorithms and processes described herein. The processor(s) 302 can be configured to fetch and execute computer-readable instructions stored in the computer-readable media 304, which can program the processor(s) 302 to perform the functions described herein.

The computer-readable media 304 include volatile and nonvolatile memory and/or removable and non-removable media implemented in any type of technology for storage of information, such as computer-readable instructions, data structures, program modules, or other data. Such computer-readable media 304 include, but are not limited to, RAM, ROM, EEPROM, flash memory or other memory technology, optical storage, solid state storage, magnetic tape, magnetic disk storage, network attached storage, storage area networks, cloud storage, or any other medium that can be used to store the desired information and that can be accessed by a computing device. Depending on the configuration of the processing device 300, the computer-readable media 304 may be a type of computer-readable storage media and/or may be a tangible non-transitory media to the extent that when mentioned, non-transitory computer-readable media exclude media such as energy, carrier signals, electromagnetic waves, and signals per se.

The computer-readable media 304 is used to store any number of functional components that are executable by the processors 302. In many implementations, these functional components comprise instructions or programs that are executable by the processors and that, when executed, specifically configure the one or more processors 302 to perform the actions attributed above to the processing device 300. In addition, the computer-readable media 304 may store data used for performing the operations described herein.

In the illustrated example, the computer-readable media 304 further includes an EHR module 308, a Raman module 310, a learning engine 312, an identification module 316, and an AST module 318. In some embodiments, the various modules in the processing device 300 may provide improved decision support tools to clinicians, healthcare providers, and or caregivers for determining treatment decisions for patients based on the identification of infection, a dysregulated host response, or organ dysfunction or organ failure in a patient.

EHR module 308 may communicate and interface with EHR system 104 to provide the processing device 300, Raman spectroscopy device 102, and/or analyzer 108 with access to EHR data for a plurality of patients, including patient data and medical history data. In some embodiments, the EHR module 308 in processing device 300 may receive and/or access EHR data from electronic health records in EHR system 104. In some embodiments, patient data that is received from EHR system 104 at EHR module 308 may be encrypted, and may be decrypted upon receipt by the EHR module 308. In some embodiments, the EHR module 308 may monitor patient data for one or more patient variables from the EHR data for a plurality of patients. In some embodiments, by monitoring EHR data, the EHR module 308 may provide an early warning detection of a likelihood of sepsis in a patient. In some embodiments, the EHR module 308 may monitor EHR data for a plurality of patients over a predetermined period of time or periodically at a predetermined interval of time to detect if any of the patient variables in the EHR data indicate a change in condition for any of the patients in the plurality of patients. In some embodiments, changes in a patient's condition may be represented by predetermined changes in patient variables, and the changes may indicate organ dysfunction in the patient.

In some embodiments, the EHR module 308 may initiate a sepsis detection process upon detecting that one or more patient variables are greater than or equal to one or more predetermined threshold values indicating a change in a patient's condition. In some embodiments, upon detecting that the one or more patient variables are greater than or equal to the one or more predetermined threshold values, the EHR module 308 may transmit a notification to at least one of the Raman module 310, the identification module 316, and AST module 318, the notification indicating that a change in a patient's condition has been detected. The notification transmitted by the EHR module 308 may trigger at least one of the Raman module 310, the identification module 316, and or AST module 318 to perform one or more steps of a sepsis detection process. In some embodiments, the EHR module 308 may also communicate with one or more databases 110 to retrieve clinical parameters data and organ dysfunction data such as SOFA scores. The data retrieved from the one or more databases 110 may also be taken into consideration for detecting a likelihood of sepsis in a patient by the EHR module 308 and/or the Raman module 310.

The Raman module 310 may communicate with the Raman spectroscopy device 200 for processing and analysis of Raman spectral data obtained from samples of patients. In some embodiments, the Raman module 310 may receive Raman spectrum data of patient samples from the Raman spectroscopy device 200, process and analyze the Raman spectrum data, and perform a rapid sepsis detection based on the Raman spectrum data. In some embodiments, the Raman module 310 may process and analyze the Raman spectrum data to identify one or more peaks that represent a Raman signature of a sample of a patient. In some embodiments, the Raman module 310 may perform an analysis of the Raman spectrum data to identify a Raman fingerprint or signature of the pathogen and one or more biomarkers indicative of the infection and dysregulated host response in the patient. In some embodiments, the Raman module 310 may correlate the Raman fingerprint or signature of the sample to a host response signature of the sample. In some embodiments, the Raman module 310 may predict a likelihood of the patient having an infection, a likelihood of the patient having bacteremia (e.g., the presence of bacteria in the patient's bloodstream), and/or a likelihood of the patient developing sepsis based on the host response signature of the sample. The use of the Raman spectrum data by the Raman module 310 may allow the processing device 300 to provide inexpensive and highly accurate results for rapid sepsis detection and prediction in patients.

In some embodiments, the Raman module 310 in the processing device 300 may identify one or more biomarkers indicative of organ failure or organ dysfunction using the Raman spectrum data obtained from the Raman spectroscopy device 200. In some embodiments, the Raman module 310 may determine one or more variables of a SOFA score using spectroscopic data of a sample of the patient obtained from the Raman spectroscopy device 200. In some embodiments, the Raman module 310 may use SOFA scores (e.g., calculated based on Raman spectroscopic data) to determine organ dysfunction.

In order to perform the sepsis detection (e.g., for detecting organ dysfunction, dysregulated host response, and/or infection), the Raman module 310 may communicate with a learning engine 312 that includes a learning algorithm 314. In some embodiments, the learning engine 312 may be configured to train the learning algorithm 314 using Raman spectrum data, EHR data, and a sepsis status corresponding to each patient in a plurality of patients. In some embodiments, the learning engine 312 may receive a plurality of EHR data for a plurality of EHR patients from the EHR system 104 via the EHR module 308. The learning engine 312 may also receive a plurality of Raman spectra data generated by the Raman spectroscopy device 102, 200 via the Raman module 310. In addition to the EHR data and the Raman data, the learning engine 312 may also receive a plurality of sepsis status determinations from the EHR system 104 via the EHR module 308, in which each sepsis status determination corresponds to a corresponding patient in the plurality of patients. In some embodiments, the learning engine 312 may train the learning algorithm 314 using the plurality of EHR data, the plurality of Raman spectra data, and the plurality of sepsis status determinations, in which the learning algorithm 314 is trained to identify a likelihood of infection or sepsis in future patients based on a classification of each EHR data and each Raman spectrum data.

In some embodiments, the EHR module 308 and/or the Raman module 310 may communicate with the learning engine 312 to train the learning algorithm 314 and apply machine learning technology to the EHR data and/or Raman spectral data for identifying sepsis in future patients based on the trained learning algorithm 314. In some embodiments, the learning algorithm 314 may be trained to identify a likelihood of infection, or sepsis in future patients based on a classification of each EHR data and each Raman spectrum data. In some embodiments, the learning algorithm 314 may be referred to herein as a learning model and/or a sepsis prediction model. In some embodiments, the learning algorithm 314 may comprise any learning algorithm, such as a Bayesian network, a neural network, a deep machine-learning algorithm, or the like. In some embodiments, the Raman module 310 and/or EHR module 308 may implement the learning algorithm 314 to assign a classification of sepsis or non-sepsis to patients based on Raman spectrum data and/or EHR data corresponding to the patients. In some embodiments, the Raman module 310 may acquire one or more patient variables from EHR data for a patient (e.g., from EHR module 308 and/or EHR system 108), receive Raman spectrum data for a sample of the patient (e.g., from the Raman spectroscopy device 102, 200), and classify the patient in an immune profile group by applying the trained learning algorithm 314 to at least one of the acquired EHR data and the Raman spectrum data.

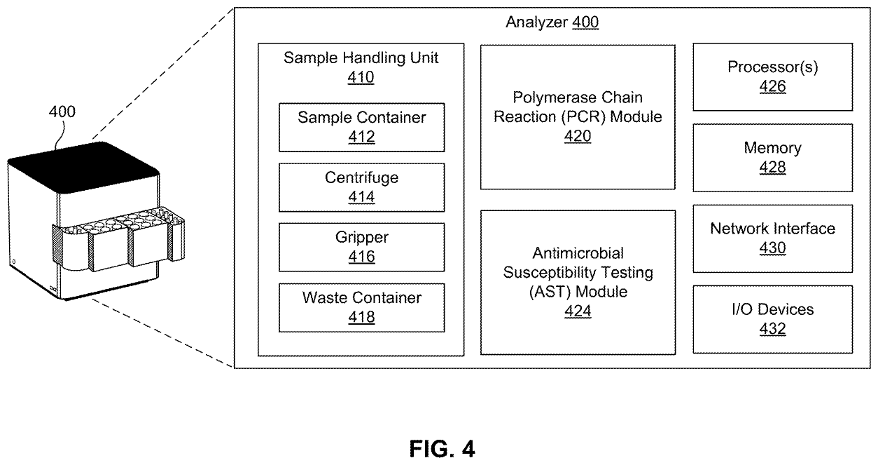

In addition to using the Raman module 310 and/or EHR module 308 to stratify patients by their sepsis status, the processing device 300 may further include identification module 316 and AST module 318 for managing PCR testing and AST testing of patient samples by the analyzer 108. In some embodiments, the identification module 316 may communicate with the analyzer 108 to initiate a PCR assay to identify a pathogen in a patient sample. In some embodiments, the identification module 316 may transmit a notification to a corresponding module in the analyzer 108 (e.g., PCR module 420 in FIG. 4), and the notification may trigger the analyzer 108 to initiate PCR for pathogen identification using the corresponding module.

In some embodiments, the identification module 316 may transmit the notification to the analyzer 108 for performing PCR after receiving data regarding a determination of a high likelihood of sepsis in the patient from the Raman module 310 and/or EHR module 308. In some embodiments, the identification module 316 might not transmit the notification to perform PCR to the analyzer 108 unless the likelihood of sepsis in a patient determined by the Raman module 310 and/or EHR module 308 is greater than or equal to a predetermined threshold value or within a predetermined range. In some embodiments, the identification module 316 may receive results from the analyzer 108 regarding an identified pathogen in the patient, and the identification module 316 may communicate with the AST module 318.

In some embodiments, the AST module 318 may receive data regarding the identified pathogen from the identification module 316, and the AST module 318 may communicate with the analyzer 108 to perform susceptibility testing of the identified pathogen. The AST module 318 may transmit a notification to a corresponding module in the analyzer 108 (e.g., AST module 424 in FIG. 4), and the notification may trigger the analyzer 108 to initiate susceptibility testing of the identified pathogen using the corresponding module. In some embodiments, the AST module 318 may receive results of the susceptibility testing from the analyzer 108, in which the results may indicate whether the identified pathogen was sensitive or resistant to one or more antimicrobials and/or antibiotics.

In some embodiments, the AST module 318 may access one or more databases 110 for epidemiology information or antibiotic resistance information for the pathogen, and generate a recommendation for treatment of a patient based on at least one of the results of the susceptibility testing and the epidemiology information or antibiotic resistance information for the pathogen. In some embodiments, the AST module 318 may also communicate with EHR module 308 to incorporate a patient's immune profile data or EHR data from EHR system 104 with the results of the susceptibility testing to determine which antimicrobials may be most effective for the patient. In some embodiments, various antimicrobials may be less effective for certain patient populations in comparison to other patient populations. Thus, the AST module 318 may take into consideration the patient's EHR data when generating the recommendation for treatment. In other words, the AST module 318 may generate the recommendation for treatment of the patient based on at least one of the EHR data of the patient, an identification of a likelihood of infection and/or sepsis in the patient (e.g., as determined by the EHR module 308 and/or Raman module 310), the identified pathogen (e.g., as received from the identification module 316 and analyzer 108), and the antibiotic susceptibility and/or antimicrobial resistance results from the analyzer 108.

Additional functional components stored in the computer-readable media 304 include an operating system 330 for controlling and managing various functions of the processing device 300. The processing device 300 also includes or maintains other functional components and data, such as other modules and data, which include programs, drivers, and the like, and the data used or generated by the functional components. Further, the processing device 300 includes many other logical, programmatic and physical components, of which those described above are merely examples that are related to the discussion herein.

The communication interface(s) 306 include one or more interfaces and hardware components for enabling communication with various other devices, including Raman spectroscopy device 102, electronic health record (EHR) system 104, analyzer 108, and a plurality of databases 110 over network 112. For example, communication interface(s) 206 facilitate communication through one or more of the Internet, cable networks, cellular networks, wireless networks (e.g., Wi-Fi, cellular) and wired networks. As several examples, the processing device 300 and other devices communicate and interact with one another using any combination of suitable communication and networking protocols, such as Internet protocol (IP), transmission control protocol (TCP), hypertext transfer protocol (HTTP), cellular or radio communication protocols, and so forth. Examples of communication interface(s) include a modem, a network interface (such as an Ethernet card), a communications port, a Personal Computer Memory Card International Association (PCMCIA) slot and card, and the like.

### Analyzer Device:

FIG. 4 illustrates an example diagram of an analyzer 400 in the sepsis detection system, according to embodiments of the present disclosure. Analyzer 400 represents an exemplary embodiment of analyzer 108 in FIG. 1B. In some embodiments, analyzer 400 may be referred to herein as an analyzer device or apparatus. Analyzer 400 may comprise a sample handling unit 410, a PCR module 420, an AST module 424, one or more processors 426, a memory 428, a network interface 430, and input/output (I/O) devices 432.

The sample handling unit 410 may include components configured to handle and/or process a sample for testing by the PCR module 420 and AST module 424. Sample handling unit 410 includes a sample container 412, centrifuge 414, gripper 416 and waste container 418. The sample container 412 may be used to hold a sample (e.g., whole blood or plasma) of a patient. In some embodiments, a user of the analyzer 400 may load at least a portion of a patient sample (e.g., by pipetting) into the sample container 412, and the user may insert the sample container 412 into the analyzer 400 for testing. In some embodiments, sample container 412 may be referred to herein as a consumable or cartridge. In some embodiments, the sample container 412 may comprise a plurality of wells that allow for testing of multiple samples or multiple antibiotics concurrently. In some embodiments, the plurality of wells in the sample container 412 are designed specifically to allow separation of a sample in different wells for PCR and AST testing by the PCR module 420 and AST module 424, respectively. In some embodiments, the sample container 412 may include one or more reagents that interact with the sample when the sample container 412 is inserted in the analyzer 400. In other embodiments, the sample handling unit 410 in the analyzer 400 includes a reagent container storing reagents that can be added to the sample in the sample container 412. In some embodiments, the reagent container may be separate from the sample container 412 and may include materials (e.g., reagents, buffers, etc.) used for PCR and/or AST. In some embodiments, the sample container 412 may be a test tube, such as a blood collection tube, a blood collection tube with a vacuum seal inside the tube, or the like. In some embodiments, the sample may be transferred at the beginning of the preparation process to a secondary container, where reagents stored in a reagent container are added or removed during the sample preparation process.

In some embodiments, each well in the plurality of wells in the sample container 412 may be connected to a corresponding reaction chamber in a plurality of reaction chambers that allow space for amplification and detection of nucleic acids in samples using PCR. In some embodiments, the sample container 412 may be manufactured with a plurality of reservoirs, one or more channels for eluate recovery, a spin column for filtering, and/or a septum for need liquid transfers. In some embodiments, the sample container 412 may also include a rotation preparation element for extraction and filtering of samples, as well as a molded lid formed from a thermoplastic elastomer (TPE) in order to allow for opening and closing of the wells and reaction chambers in the sample container 412. In some embodiments, some wells of the sample container 412 may include beads for cell lysis, or magnetic beads to extract nucleic acids.

In some embodiments, the sample handling unit 410 may include a built-in centrifuge 414 within the analyzer 400. The centrifuge 414 may allow separation of one or more samples in the sample container 412 in order to process the sample or to isolate nucleic acids for detection. In addition to the centrifuge 414, the sample handling unit 410 may also include a pipetting system (not shown) to allow multifunctional interactions between the sample container 412 and the components in the analyzer 400. In some embodiments, the pipetting system of the sample handling unit 410 may support the steps for PCR-based pathogen identification and growth-based AST by the PCR module 420 and AST module 424, respectively.

The sample handling unit 410 may further comprise a gripper 416. The gripper 416 may allow movement of the sample container 412 between different components in the sample handling unit 410, including movement of the sample container 412 to and from the PCR module 420 and AST module. In some embodiments, the gripper 416 may prevent slippage of the sample container 412 and may provide a firm grip for holding the sample container 412 in place within different components in the analyzer 400. In addition to the gripper 416, the sample handling unit 410 may include other components that allow the sample container 412 to be moved by an automated process within the analyzer 400, such that the sample container 412 may interact with the PCR module 420 and AST module 424 for testing.

The sample handling unit 412 may also include a waste container 418. The waste container 418 may be configured to hold any waste liquids or unreacted materials resulting from the PCR reactions and susceptibility testing of samples. In some embodiments, the sample container 412, centrifuge 414, gripper 418, and waste container 418 may be coupled together for proper handling of a sample to engage with the PCR module 420 and AST module 424. In some embodiments, the sample handling unit 412 may be configured to receive a sample of a patient using the sample container 412, and at least a portion of the sample may be processed using at least one of the centrifuge 414 and the waste container 418. The sample container 412 may be handled using the gripper 418. In some embodiments, operations of the centrifuge 414, gripper 418, and any other components in the sample handling unit 410 may be controlled by one or more processors 426 in the analyzer 400.

In some embodiments, the analyzer 400 may include whole blood collection and sample processing devices that allow separation of pathogenic cells for clean and sensitive PCR or direct from blood rapid AST at the cellular level. In some embodiments, the sample processing devices of the analyzer 400 may utilize antimicrobial neutralization technology to assure that the pathogenic bacteria in a blood sample is unaffected by antibiotics that may have been in the bloodstream of a patient, and in order to prevent adverse effects on the results from the analyzer 400. In some embodiments, the PCR module 420 and the AST module 424 may be communicatively coupled to the sample handling unit 410 and may utilize the same sample container 412 for pathogen detection and susceptibility testing. In some embodiments, the sample container 412 may comprise a tube that is multifunctional, supporting both preparation for PCR and growth-based microbiology AST.

In some embodiments, the PCR module 420 may perform real-time PCR of patient samples for pathogen identification directly from a blood sample of a patient. In some embodiments, the PCR module 420 may include one or more components used for performing PCR, including a thermal cycler configured to control temperatures during PCR, an optical system for collecting data from the plurality of wells in the sample container 412, reaction modules, and the like. In some embodiments, the PCR module 420 may be configured to receive a sample of a patient in the sample container 412 from the sample handling unit 410. The PCR module 420 may amplify a nucleic acid within the sample of the patient using a PCR process, detect the amplified nucleic acid (e.g., a product nucleic acid), and identify a pathogen present in the sample of the patient based on the detected amplified nucleic acid. In some embodiments, the PCR module 420 may detect a product nucleic acid and identify a pathogen directly from the product nucleic acid. In additional or alternative embodiments, the PCR module 420 may detect a product nucleic acid and identify a transcriptomic product indicative of an infection in a patient, in which the the transcriptomic product includes RNA. In additional or alternative embodiments, the PCR module 420 may include different areas that are exposed to different cycling conditions, so that several reactions can be performed with different cycling conditions in parallel.

After pathogen identification, the PCR module 420 may communicate with the AST module 424, such as by transmitting a command or indication to perform an AST assay. The AST module 424 may be configured to perform susceptibility testing of an identified pathogen. After pathogen identification (e.g., by the PCR module 420 in the analyzer 400, and/or by the processing device 106), the AST module 424 may receive the sample container 412 with the plurality of wells containing various concentrations of different antibiotics or antimicrobials for performing different susceptibility tests for the identified pathogen. The AST module 424 may expose samples of the pathogen to the different antibiotic or antimicrobial in each well.

The AST module 424 may then process results from the sample container 412 and determine to which antibiotics or antimicrobials the pathogen (e.g., infection in the patient) is susceptible, intermediate, or resistant (S/I/R). In some embodiments, the AST module 424 may perform an enrichment step of a sample containing the pathogen prior to performing an AST assay. In some embodiments, the AST module 424 may identify a minimum inhibitory concentration (MIC) of an antibiotic that inhibits growth of a bacteria or pathogen. In some embodiments, the AST module 424 may perform rapid direct-from-blood antimicrobial susceptibility testing using single cell microscopy and specialty stains. In some embodiments, the AST module 424 may perform antimicrobial susceptibility testing of the pathogen by enriching a sample of the patient with the pathogen, distributing aliquots of the enriched sample to the plurality of wells in the sample container 412, and performing a single-cell microscopic AST assay of the pathogen in the patient.

In some embodiments, the AST module 424 may include a microscope configured to perform single cell microscopy of the sample in the sample container 412. In some embodiments, by using single cell microscopy, the AST module 424 may provide a faster result or enhance the ability to detect resistance mechanisms by monitoring patterns created by grown microorganisms, such as filaments or chains. In some embodiments, the microscope in the AST module 424 may acquire images of single microorganisms and identify an antimicrobial phenotypical resistance of the microorganisms (e.g., pathogens) based on the acquired images, leading to a diagnostic pathway for treatment. In some embodiments, the AST module 424 may also utilize PCR techniques to detect genotypic resistance information of a pathogen. In some embodiments, the AST workflow of the AST module 424 may include preparing various concentrations of isolated pathogens and/or concentrations of different antibiotics or antimicrobials, enrichment and clean-up of the sample, execution of a phenotypic antibiotic susceptibility test, putting the pathogens in contact with antibiotics, and monitoring growth.

In some embodiments, the PCR module 420 and the AST module 424 may use a single sample container 412 or different sample containers 412 for performing pathogen identification and AST. In some embodiments, the sample container 412 may be manufactured and engineered to provide efficient cleanup, concentration separations, cell preparation, cell debris removal and the like for rapid direct-from-blood identification, along with concentration and pathogen viability maintenance for rapid AST.

Analyzer 400 further includes one or more processors 426. Each processor 426 is a single processing unit or a number of processing units, and may include single or multiple computing units or multiple processing cores. The processor(s) 426 can be implemented as one or more microprocessors, microcomputers, microcontrollers, digital signal processors, central processing units, state machines, logic circuitries, and/or any devices that manipulate signals based on operational instructions. For instance, the processor(s) 426 may be one or more hardware processors and/or logic circuits of any suitable type specifically programmed or configured to execute the algorithms and processes described herein. The processor(s) 426 can be configured to fetch and execute computer-readable instructions stored in the memory 428, which can program the processor(s) 426 to perform the functions described herein.

In some embodiments, memory 428 may represent a computer-readable media that may include volatile and nonvolatile memory and/or removable and non-removable media implemented in any type of technology for storage of information, such as computer-readable instructions, data structures, program modules, or other data. Such computer-readable media include, but are not limited to, RAM, ROM, EEPROM, flash memory or other memory technology, optical storage, solid state storage, magnetic tape, magnetic disk storage, network attached storage, storage area networks, cloud storage, or any other medium that can be used to store the desired information and that can be accessed by a computing device. Depending on the configuration of the analyzer 400, the computer-readable media may be a type of computer-readable storage media and/or may be a tangible non-transitory media to the extent that when mentioned, non-transitory computer-readable media exclude media such as energy, carrier signals, electromagnetic waves, and signals per se.

The computer-readable media may be used to store any number of functional components that are executable by the processors 426. In many implementations, these functional components comprise instructions or programs that are executable by the processors and that, when executed, specifically configure the one or more processors 426 to perform the actions described herein. In addition, the computer-readable media may store data used for performing the operations described herein below.

Network interface 430 includes one or more interfaces and hardware components for enabling communication with various other devices in the sepsis detection system 101, such as the Raman spectroscopy device 102 or 200, EHR system 104, processing device 106 or 300, and/or the plurality of databases 110 over network 112. For example, network interface 430 facilitates communication through one or more of the Internet, cable networks, cellular networks, wireless networks (e.g., Wi-Fi, cellular) and wired networks. In some embodiments, analyzer 400 and processing device 106 or 300 communicate and interact with one another using any combination of suitable communication and networking protocols, such as Internet protocol (IP), transmission control protocol (TCP), hypertext transfer protocol (HTTP), cellular or radio communication protocols, and so forth. Examples of communication interface(s) include a modem, a network interface (such as an Ethernet card), a communications port, a PCMCIA slot and card, and the like.

Analyzer 400 may be equipped with various input/output (I/O) devices 432. Such I/O devices 432 may include various user interface controls, such as buttons, joystick, keyboard, mouse, display, touch screen, and the like, connection ports, and so forth. Additionally, analyzer 400 may include various other components that are not shown, examples of which include removable storage, a power source, such as a battery and power control unit, and so forth. Analyzer 400 may also include or maintain other functional components and data, such as other modules and data, which include programs, drivers, and the like, and the data used or generated by the functional components. Further, analyzer 400 may include many other logical, programmatic and physical components, of which those described above are merely examples that are related to the discussion herein.

### Example Methods of Operation:

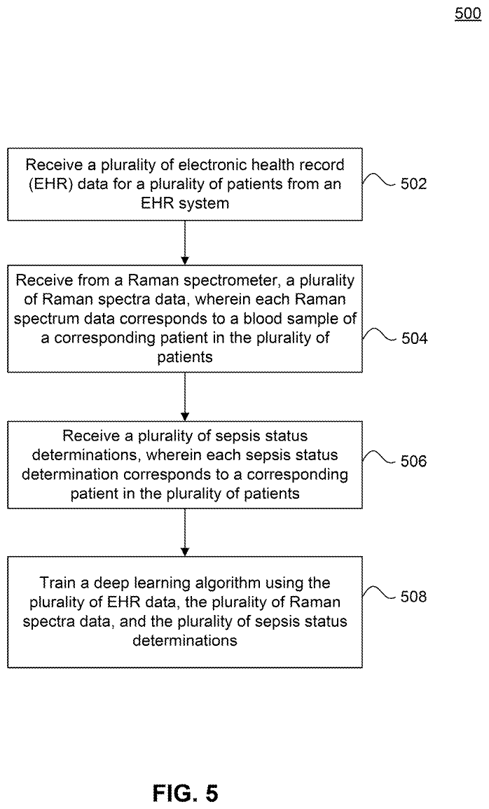

FIG. 5 illustrates an example flowchart diagram of a method 500 for training a learning algorithm for identifying sepsis in patients, according to embodiments of the present disclosure. The steps of method 500 may be performed by processing device 106 or 300 in the sepsis detection system 101.

Method 500 of FIG. 5 begins with step 502, in which a plurality of electronic health record (EHR) data for a plurality of patients from an EHR system is received. In some embodiments, the processing device 300 may receive EHR data for patients from the EHR system 104. At step 504, a plurality of Raman spectra data is received from a Raman spectrometer. In some embodiments, the processing device 300 may receive Raman spectra data from the Raman spectroscopy device 102 or 200. In some embodiments, each Raman spectrum data corresponds to a blood sample of a corresponding patient in the plurality of patients.

At step 506, a plurality of sepsis status determinations may be received. In some embodiments, the processing device 300 may receive data regarding sepsis status determinations from at least one of the one or more databases 110, the EHR system 104, or the Raman spectroscopy device 102, 200. In some embodiments, each sepsis status determination corresponds to a corresponding patient in the plurality of patients.

At step 508, a deep learning algorithm is trained using the plurality of EHR data, the plurality of Raman spectra data, and the plurality of sepsis status determinations. In some embodiments, the learning engine 312 in processing device 300 may train the learning algorithm 314 to identify a likelihood of infection or sepsis in future patients based on a classification of each EHR data and each Raman spectrum data.

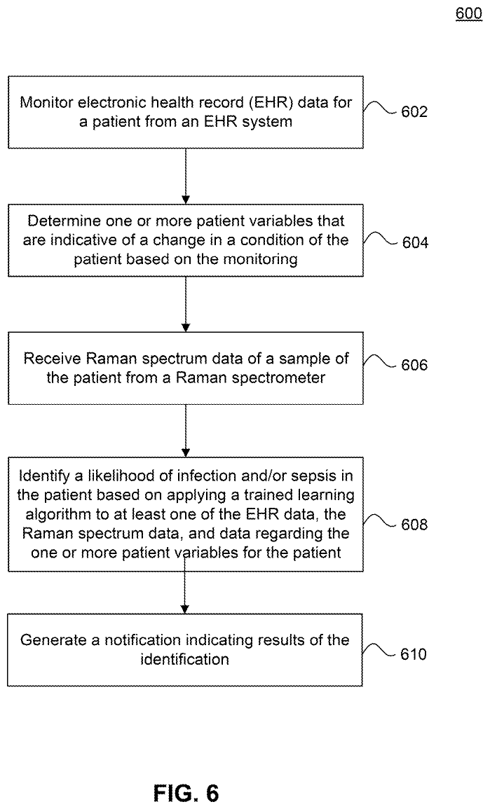

FIG. 6 illustrates an example flowchart diagram of a method 600 for determining a likelihood of sepsis in a patient, according to embodiments of the present disclosure. The steps of method 600 may be performed by processing device 106 or 300 in the sepsis detection system 101.

Method 600 of FIG. 6 begins with step 602, in which electronic health record (EHR) data for a patient from an EHR system is monitored. In some embodiments, the processing device 300 may monitor and/or acquire EHR data for a patient from the EHR system 104. At step 604, one or more patient variables that are indicative of a change in a condition of the patient may be determined based on the monitoring. In some embodiments, the processing device 300 may determine one or more patient variables indicative of a change in the patient's based on the monitoring.

At step 606, Raman spectrum data of a sample of the patient may be received from a Raman spectrometer. In some embodiments, the processing device 300 may receive Raman spectrum data of the patient's sample from the Raman spectroscopy device 102 or 200. At step 608, a likelihood of infection and/or sepsis in the patient may be identified based on applying a trained learning algorithm to the collected EHR data, the Raman data, and/or patient variable data. In some embodiments, the processing device 300 may identify a likelihood of infection and/or sepsis in the patient based on applying the trained learning algorithm 314 (e.g., after training by the learning engine 312) to at least one of the EHR data, the Raman spectrum data, and data regarding the one or more patient variables for the patient.

At step 610, a notification indicating results of the identification may be generated. In some embodiments, the processing device 300 may generate a notification indicating results of the identification and transmit the notification to a device associated with a caregiver or healthcare provider of the patient.

### Example Computer System:

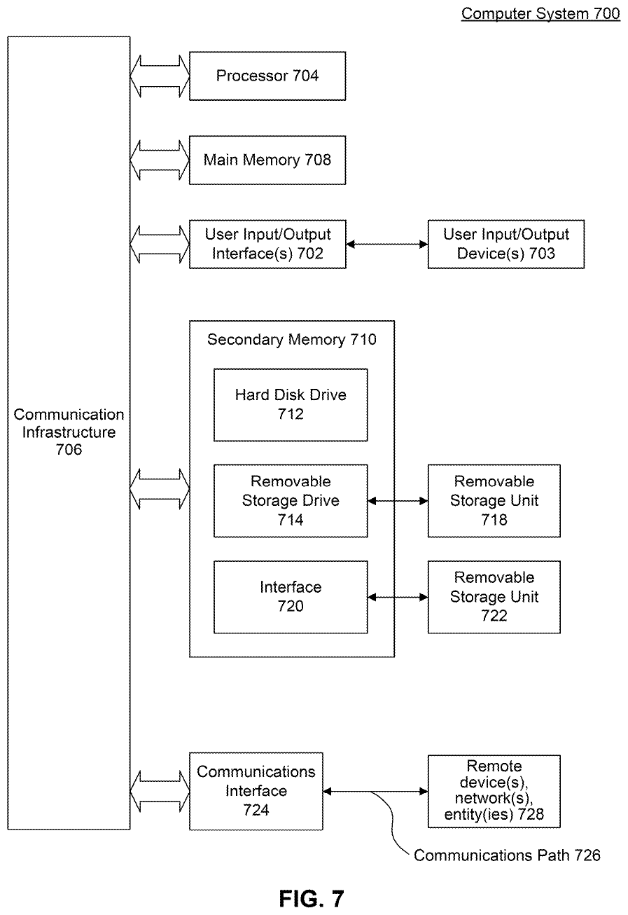

FIG. 7 is a block diagram of example components of computer system 700. One or more computer systems 700 may be used, for example, to implement any of the embodiments discussed herein, as well as combinations and sub-combinations thereof. In some embodiments, one or more computer systems 700 may be used to implement the methods 500 and 600 shown in FIGS. 5 and 6, respectively, processing device 106, 300, analyzer 108, 400, Raman spectroscopy device 102, 200, and EHR system 104 shown in FIGS. 1B and 2-4, as described herein. Computer system 700 may include one or more processors (also called central processing units, or CPUs), such as a processor 704. Processor 704 may be connected to a communication infrastructure or bus 706.

Computer system 700 may also include user input/output interface(s) 702, such as monitors, keyboards, pointing devices, etc., which may communicate with communication infrastructure 706 through user input/output device(s) 703.

One or more of processors 704 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 may also include a main or primary memory 708, such as random access memory (RAM). Main memory 708 may include one or more levels of cache. Main memory 708 may have stored therein control logic (i.e., computer software) and/or data. In some embodiments, main memory 708 may include optical logic configured to perform sepsis detection, sepsis likelihood prediction, pathogen identification, and susceptibility testing, and generate recommendations for treatment of patients accordingly.

Computer system 700 may also include one or more secondary storage devices or memory 710. Secondary memory 710 may include, for example, a hard disk drive 712 and/or a removable storage drive 714.

Removable storage drive 714 may interact with a removable storage unit 718. Removable storage unit 718 may include a computer usable or readable storage device having stored thereon computer software (control logic) and/or data. Removable storage unit 718 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 714 may read from and/or write to removable storage unit 718.

Secondary memory 710 may include other means, devices, components, instrumentalities or other approaches for allowing computer programs and/or other instructions and/or data to be accessed by computer system 700. Such means, devices, components, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 700 may further include a communication or network interface 724. Communication interface 724 may enable computer system 700 to communicate and interact with any combination of external devices, external networks, external entities, etc. (individually and collectively referenced by reference number 728). For example, communication interface 724 may allow computer system 700 to communicate with external or remote devices 728 over communications path 726, which may be wired and/or wireless (or a combination thereof), and which may include any combination of LANs, WANs, the Internet, etc. Control logic and/or data may be transmitted to and from computer system 700 via communication path 726.

Computer system 700 may also be any of a personal digital assistant (PDA), desktop workstation, laptop or notebook computer, netbook, tablet, smartphone, smartwatch or other wearables, appliance, part of the Internet-of-Things, and/or embedded system, to name a few non-limiting examples, or any combination thereof.

Computer system 700 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 700 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 7. In particular, embodiments can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

Embodiments of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

1. A system comprising:
a first subsystem configured to detect a presence of an infection in a patient;
a second subsystem configured to detect a presence of a dysregulated host response in the patient;
a third subsystem configured to detect organ dysfunction in the patient; and
a processing device, wherein the first subsystem, the second subsystem, the third subsystem, and the processing device are communicatively coupled together via a network, and
wherein the processing device is configured to:
determine a presence of sepsis in the patient based on the presence of the infection, the presence of the dysregulated host response, and clinical data indicative of the organ dysfunction in the patient.

2. The system of claim 1, wherein the first subsystem is configured to detect the infection in the patient and comprises:
a polymerase chain reaction (PCR) module configured to perform PCR processing of a first sample of the patient and detect a product nucleic acid; and
an identification module configured to identify a pathogen directly from the product nucleic acid.

3. The system of claim 1, wherein the presence of the infection in the first subsystem is determined using spectroscopic data of a first sample of the patient obtained from a Raman spectrometer.

4. The system of claim 1, wherein the presence of the infection is determined by an analysis of data measured from a pathogen causing the infection in the patient.

5. The system of claim 1, wherein the presence of the infection is determined by an analysis of data related to an immune response of the patient.

6. The system of claim 1, wherein the presence of the dysregulated host response in the second subsystem is determined using spectroscopic data of a first sample of the patient obtained from a Raman spectrometer.

7. The system of claim 1, wherein the second subsystem comprises:
a Raman spectrometer configured to obtain Raman spectrum data of a first sample of the patient; and
a processor configured to analyze the Raman spectrum data to identify one or more signals indicative of the dysregulated host response in the patient.

8. The system of claim 1, wherein the second subsystem comprises:
a polymerase chain reaction (PCR) module configured to perform PCR processing of a first sample of the patient and detect a product nucleic acid; and
an identification module configured to identify a transcriptomic product indicative of the dysregulated host response in the patient, the transcriptomic product comprising RNA.

9. The system of claim 1, wherein the third subsystem is further configured to:
collect the clinical data indicative of the organ dysfunction in the patient from at least one of an electronic health record of the patient and one or more databases.

10. The system of claim 10, wherein the clinical data indicative of the organ dysfunction in the patient comprises one or more scores associated with at least one of sequential organ failure assessment (SOFA), quick SOFA, national early warning score (NEWS), vital signs of the patient, and biomarkers of the patient.

11. The system of claim 1, wherein the organ dysfunction in the third subsystem is determined using spectroscopic data of a first sample of the patient obtained from a Raman spectrometer.

12. The system of claim 1, wherein the organ dysfunction in the third subsystem is determined using a combination of the clinical data and spectroscopic data of a first sample of the patient obtained from a Raman spectrometer.

13. The system of claim 1, wherein the organ dysfunction in the third subsystem is determined using a sequential organ failure assessment (SOFA) score, and wherein at least one or more variables of the SOFA score is determined using spectroscopic data of a first sample of the patient obtained from a Raman spectrometer.

14. A system comprising:
a first subsystem configured to detect a presence of an infection in a patient;
a second subsystem configured to detect a presence of a dysregulated host response in the patient;
a third subsystem configured to detect organ dysfunction in the patient;
a fourth subsystem configured to detect antimicrobial resistance (AMR) of a pathogen in the patient from a sample; and
a processing device, wherein the first subsystem, the second subsystem, the third subsystem, the fourth subsystem, and the processing device are communicatively coupled together via a network, and
wherein the processing device is configured to:
determine a presence of sepsis in the patient based on the presence of the infection, the presence of the dysregulated host response, and clinical data indicative of the organ dysfunction in the patient.

15. The system of claim 15, wherein the AMR of the pathogen is determined by genotypic information obtained from a PCR targeting one or more resistance genes of the pathogen.

16. The system of claim 15, wherein the AMR of the pathogen is determined by phenotypic information obtained by an antimicrobial susceptibility testing (AST) module configured to perform an AST assay of the pathogen in the patient.

17. The system of claim 17, wherein the AST module comprises a microscope configured to acquire one or more images of the pathogen, and wherein the pathogen comprises a single-cell microorganism.

18. The system of claim 17, wherein the first subsystem comprises an identification module configured to identify the pathogen in the patient from the sample, and wherein the processing device is further configured to:
receive data regarding the pathogen from the identification module and results of the AST assay from the AST module;
access one or more databases for epidemiology information or antibiotic resistance information for the pathogen; and
generate a recommendation for treatment of the patient based on the results of the AST assay and the epidemiology information or antibiotic resistance information for the pathogen from the one or more databases.

19. The system of claim 19, wherein the processing device is further configured to:
access an electronic health record (EHR) of the patient to identify an immune profile of the patient; and
generate the recommendation for treatment of the patient further based on the immune profile of the patient.

20. A system comprising:
a first subsystem configured to receive a first sample of the patient;
a second subsystem configured to obtain Raman spectrum data of the patient from the first sample;
a processing device configured to:
acquire one or more patient variables from electronic health record (EHR) data for the patient;
receive the Raman spectrum data of the patient from the second subsystem; and classify the patient in an immune profile group by applying a trained learning algorithm to at least one of the EHR data and the Raman spectrum data.

21. The system of claim 21, wherein the one or more patient variables comprise at least one of temperature, heart rate, systolic blood pressure, respiratory rate, white blood cell count, platelet count, ratio of arterial oxygen partial pressure to fractional inspired oxygen (PaO2/FiO2), bilirubin level, Glasgow Coma Scale score, cardiovascular mean arterial pressure, creatinine level, lactate level, C-reactive protein level, and procalcitonin level.

22. The system of claim 21, wherein the processing device is further configured to:
identify a likelihood of infection and/or sepsis in the patient based on applying the trained learning algorithm to at least one of the EHR data, the Raman spectrum data, and data regarding the one or more patient variables for the patient; and
generate a notification indicating results of the identification.

23. The system of claim 23, wherein the results of the identification indicate a low likelihood of infection and/or sepsis in the patient, wherein a value of the likelihood is less than a predetermined threshold value.

24. The system of claim 23, wherein the results of the identification indicate a high likelihood of infection and/or sepsis in the patient, wherein a value of the likelihood is greater than or equal to a predetermined threshold value.

25. The system of claim 23, wherein the processing device is further configured to: generate a recommendation for a treatment for the patient based on the results of the identification, wherein the recommendation for the treatment is based on antibiotic susceptibility testing (AST) of a sample of the patient.

26. The system of claim 1, wherein the first subsystem is configured to detect the infection in the patient and comprises:
a polymerase chain reaction (PCR) module configured to perform PCR processing of a first sample and detect a product nucleic acid; and
an identification module configured to identify a transcriptomic product indicative of the infection in the patient, the transcriptomic product comprising RNA.
